(19) United States
(12) Patent Application Publication
(10) Pub. No.: US 2021/0085332 A1
(43) Pub. Date: Mar. 25, 2021

(54) SYSTEM AND METHOD FOR UNSHEATHING AND RESHEATHING AN EMBOLIC DEVICE DELIVERY SYSTEM

(71) Applicant: AVANTEC VASCULAR CORPORATION, Sunnyvale, CA (US)

(72) Inventors: SITH KHOUNE, Sunnyvale, CA (US); Hidenori Yamaguchi, Sunnyvale, CA (US)

(21) Appl. No.: 16/577,209

(22) Filed: Sep. 20, 2019

(51) Int. Cl. A61B 17/12; A61M 25/06

(52) U.S. Cl. CPC A61B 17/12113; A61B 2017/00477; A61M 2025/0681; A61M 25/0662

(57) ABSTRACT

A system and method for sheathing and resheathing a delivery catheter for embolic device are disclosed. An embodiment is directed to an embolic device delivery system that includes a delivery catheter having an embolic device coupler, a locking tube disposed about the delivery catheter, a protective sheath, and a sheath outer component disposed about the protective sheath configured to unsheathe and resheathe the delivery catheter, and smooth transferring of the embolic device into a microcatheter. In one embodiment, the coupler may be an elongated shape having a loop portion for engaging the coil toward the distal end of the delivery catheter. The loop portion of the coupler may be an elastic material or a shape-memory alloy, such as Nitinol or nickel titanium, such that the loop portion is bendable at various angles. Together, the delivery catheter and coupler may be inserted into an artery and carry an embolic device to an aneurysm for placement near an aneurysm.

### BACKGROUND

An aneurysm is a blood bulge formed in a wall of an artery and can develop in any artery, including brain, aorta, legs, and spleen. Various aneurysms are typically formed in a saccular form and if the saccular aneurysm ruptures, a stroke, also known as a subarachnoid hemorrhage, may occur. Open surgery to clip or seal the aneurysm is an option for treating and removing an aneurysm; however, the surgery often carries risks and may be inappropriate or dangerous for larger sizes of aneurysms and/or aneurysms in more sensitive locations. Therefore, treating, reducing, and/or removing aneurysms is important to the long-term health of patients.

As an alternative to open surgery, a surgeon may perform a minimally invasive procedure whereby an occlusion embolic device is placed within an artery in an effort to treat the developed aneurysm. In such a procedure, the occlusion embolic device (e.g., a blocking device) is placed into the saccular aneurysm at a position to isolate or block the saccular aneurysm from a blood vessel. The placement of the occlusion embolic device is typically accomplished using a catheter carrying the occlusion embolic device such that the device may be inserted into a blood vessel and steered through the blood vessel to treat the aneurysm.

Conventional embolic device deployment systems exhibit difficulties with respect to embolic device placement as maneuvering, placing and releasing the embolic device within an artery inside a patient's body and are proven to be cumbersome. This is especially true for brain aneurysms as the deployment procedure requires accurate placement of the embolic device and any error during the procedure may result in significant damage to the brain.

### BRIEF DESCRIPTION OF THE DRAWINGS

Aspects and many of the attendant advantages of the claims will become more readily appreciated as the same become better understood by reference to the following detailed description, when taken in conjunction with the accompanying drawings, wherein:

FIG. 1(a) is a perspective diagram of a patient with a brain aneurysm;

FIG. 1(b) shows the brain aneurysm of FIG. 1(a) in greater detail during treatment;

FIG. 2 shows a diagram of a portion of an embolic device delivery system showing a catheter in a state of resheathing/unsheathing according to an embodiment of the subject matter disclosed herein;

FIG. 3 shows a more detailed diagram of a portion of an embolic device delivery system showing a catheter in a state of resheathing/unsheathing according to an embodiment of the subject matter disclosed herein;

FIGS. 4a-c are diagrams of a maneuverable tip of a coupler protruding through a lower locking window of an embolic device delivery system according to an embodiment of the subject matter disclosed herein;

FIG. 5 is a diagram of an actuator handle connected to a proximal end of an embolic device delivery system according to an embodiment of the subject matter disclosed herein; and

FIG. 6 is a flowchart for illustrating a method for sheathing and unsheathing an embolic device delivery system according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the systems and methods described herein may be practiced. The embolic device delivery system may, however, be embodied in many different forms and should not be construed as limited to the embodiments set forth herein; rather, these embodiments are provided such that this disclosure will satisfy the statutory requirements and convey the scope of the subject matter to those skilled in the art.

By way of an overview, the subject matter disclosed herein may be directed to an embolic device delivery system, method, and device. In an embodiment, e.g., an embolic device delivery system comprises a delivery catheter having an embolic device coupler disposed at one end, a locking tube disposed about the delivery catheter, a protective sheath, and a sheath outer component disposed about the protective sheath configured to unsheathe and resheathe the delivery catheter. In one embodiment, the coupler may be an elongated shape having a loop portion for engaging the coil toward the distal end of the delivery catheter. The loop portion of the coupler may be an elastic material or a shape-memory alloy, such as Nitinol or nickel titanium, such that the loop portion is bendable at various angles. Together, the delivery catheter and coupler may be inserted into an artery and carry an embolic device to an aneurysm for placement near and treatment of the aneurysm.

The embolic device may be coupled by a retaining mechanism (e.g. an aperture) to the distal end of the coupler. Until the embolic device is delivered to a certain location within an artery, the embolic device may be secured to the coupler that is disposed within the delivery catheter by interlocking the loop portion of the coupler with the retaining mechanism of the embolic device. Thus, when unsheathing the delivery catheter from the reusable sheath, the locking tube and the sheath outer component may be used to bias the protective sheath to reveal the delivery catheter for inserting into an artery. Similarly, when being retrieved, the locking tube and the sheath outer component may be used to bias the protective sheath to a closed position that encloses the delivery catheter for subsequent use. In some embodiment, the undelivered embolic device may still be attached to the coupling mechanism.

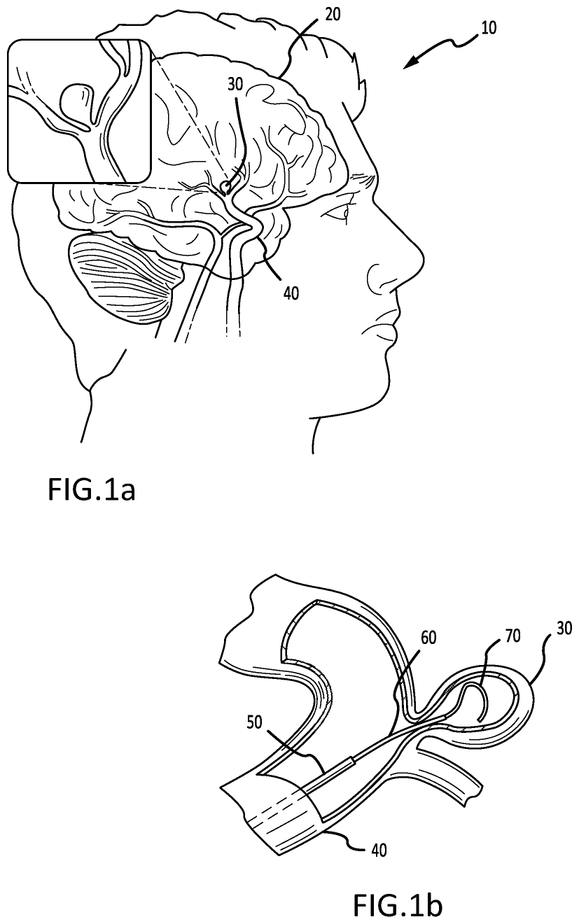

The proximal end of the coupler is coupled to an actuator such that the coupler is maneuverable by a surgeon. The actuator has a handler for a surgeon to manipulate the coupler with a mechanism to release the embolic device. When the delivery catheter reaches the desired location in the artery, the embolic device may be released from the delivery catheter by simply pulling the coupler via the actuator proximally such that the U-shaped curve portion of the coupler and the loop portion of the coupler first become disengaged from the locking windows and then the coupler releases the embolic device by disengaging the retaining mechanism. During the pulling, the loop portion of the coupler, which does not have a hook or curved end, does not pose a great risk of bumping, pulling or moving the embolic device after placement because the flexible loop end of the coupler has been at least partially straightened by the edge of the implant's retaining mechanism with or without cross bar and is more easily maneuvered from the locking windows and retaining mechanism. Further, the straightened coupler can be easily pulled through the tube of the delivery catheter. This is advantageous over conventional embolic device delivery systems that use hooks or other non-flexible engagement/delivery components to easily dislodge or move the embolic device once placed. In addition, the simple structure of the embodiments discussed herein are more efficiently manufactured with costs that are more reasonable and the reusable nature of resheathing the delivery catheter allows for multiple subsequent uses. These and other advantages will become more apparent in the detailed descriptions below with respect to FIGS. 1-6.

FIG. 1(a) is a perspective diagram of a patient 10 with a brain aneurysm 30. An aneurysm may be formed in any artery of a human body including the heart and the brain. An aneurysm that forms in blood vessels (e.g., arteries) 40 in the brain 20 is called a cerebral aneurysm or brain aneurysm 30. In this example, the brain aneurysm 30 resembles a balloon. Since the aneurysm 30 is caused due to the weakness of the artery, an aneurysm 30 with or near a thin artery wall 40 may rupture. A ruptured aneurysm (not shown) significantly contributes to the occurrence of a stroke and should be treated prior to rupture. FIG. 1(b) shows the brain aneurysm of FIG. 1(a) in greater detail and in the midst of a procedure using an embolic delivery device. The embolic device deployment system of FIG. 1(b) contains a deployment catheter 50 and, on occasion, a micro catheter 60 inside of the deployment catheter 50. The conventional embolic deployment system is inserted into the artery 40 and passed through the artery 40 to reach the desired location. At the desired location, the deployment catheter 50 and/or micro catheter 60 releases an embolic device 70 into the inside of the aneurysm 30 or near the aneurysm 30.

Depending on the location or nature of the aneurysm 30, the embolic device 70 is placed inside of the saccular aneurysm 30 as shown in FIG. 1(b) or the neck of the aneurysm to prevent further blood flow going into the aneurysm 30. The deployment mechanism of the embolic device 70 may include pushing off the embolic device 70 from the deployment catheter 50 as shown in FIG. 1(b); using a thread or fiber (not shown) for engaging the embolic device 70 and cutting off the thread or fiber when disengaging the embolic device 70; using a pressure (not shown), heat (not shown), or electricity (not shown) for releasing the embolic device 70; and unlocking an interlocking mechanism to release the embolic device 70 from the deployment catheter 50 (not shown). The interlocking mechanism of the embolic device delivery system typically interlocks an embolic device 70 with a portion of the deployment catheter 50 to carry the embolic device 70 through the inside of the artery 40 such that the deployment catheter 50 maneuvers the artery 40 with the embolic device 70 firmly coupled to the deployment catheter 50.

However, the interlocking mechanisms in conventional embolic device deployment systems require many components or features to achieve reliability, such as firmly holding the embolic device until the deployment catheter 70 reaches a desired location for effectively releasing the embolic device 70. Many conventional interlocking mechanisms require an additional locking component to interlock the deployment catheter 50 and embolic device 70 in a fixed manner. Further, if the interlocking mechanism fails to release the embolic device in the desired location, retrieving the embolic device along with the catheter for a second use is not possible with conventional embolic device delivery systems. Thus, as the catheter is removed from the artery and the patient (with or without the embolic device still attached), the catheter cannot be easily resheathed and reused with conventional systems.

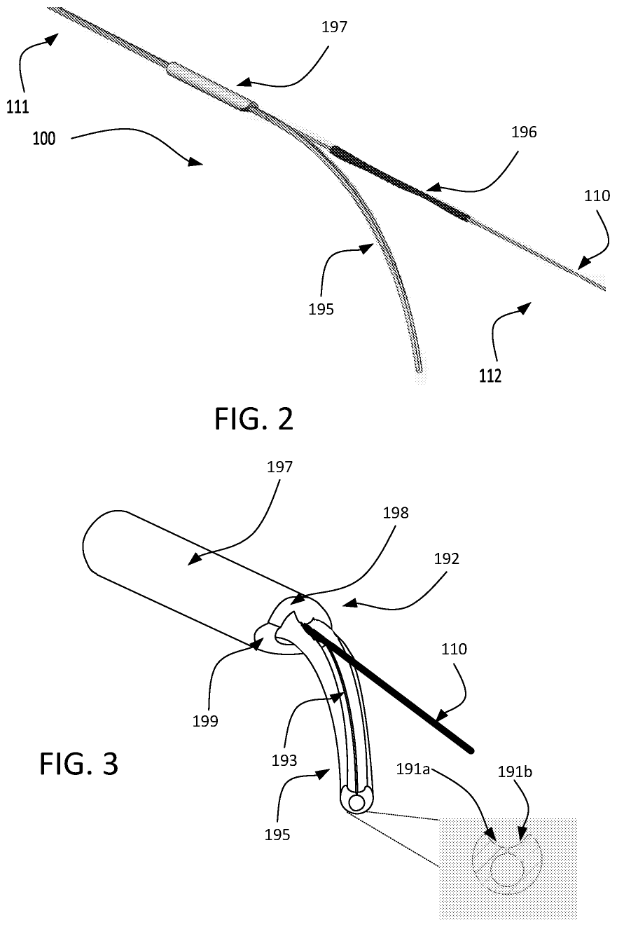

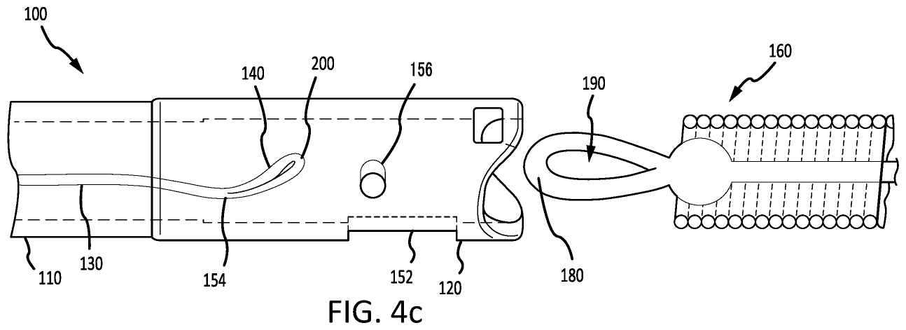

FIG. 2 shows a diagram of a portion of an embolic device delivery system 100 showing a catheter 110 in a state of resheathing/unsheathing according to an embodiment of the subject matter disclosed herein. In this view, one can see a catheter 110 that may have a coupler (not shown in FIG. 2 but shown in FIG. 4 and described below) disposed at a distal end and configured to engage an embolic device (also shown and described with respect to FIG. 4 below. The embolic device delivery system 100 further includes a reusable sheath 195 configured to surround the catheter 100 in a sheathed state (as generally shown at proximal end 111 in FIG. 2) and configured to reveal the catheter 100 in an unsheathed state (as generally shown at distal end 112 in FIG. 2). The system 100 further includes a sheath outer component 197 slidably coupled to the reusable sheath 195 and configured to bias the sheath 195 to a sheathed state surrounding the catheter 110. The system 100 further includes a locking tube 196 slidably coupled to the catheter 110 and configured to bias the catheter 110 for unsheathing from the sheath 195, and to prevent inadvertent deployment of the embolic implant

In specific, this view in FIG. 2 shows a part of an overall embolic device delivery system 100 at a mid-portion where the catheter 110 may be unsheathed or resheathed depending on current undertaken activity. That is, if the embolic device delivery system 100 is being used to deliver the embolic device (not shown), the catheter 110 may be unsheathed from a protective sheath 195 at this point. Likewise, if the catheter 110 is being retrieved either with or without the embolic device attached, FIG. 2 may be illustrating the point of resheathing the catheter 110 in the protective sheath 195. As is described below, the outer sheath component 196 and the locking tube 197 may be used to bias the catheter 110 and the sheath 195 to either the sheathed state (e.g., resheathed state) or the unsheathed state.

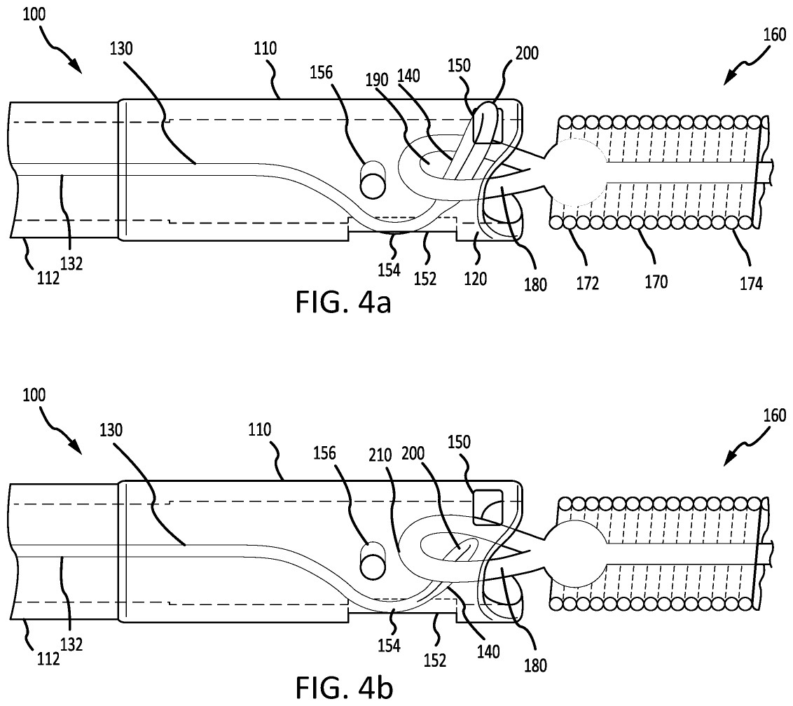

Turning to the locking tube 196 first, the locking tube 196 is slidably disposed about the catheter 110. The locking tube 197 includes a tubular cavity surrounded by a conical body that tapers at a proximal end (e.g., an end closer to the sheathe/unsheathe point). The locking tube 196 facilitates the unsheathing and resheathing of the catheter during a respective unsheathing or resheathing procedure. The locking tube 196 comprises an asymptotically decreasing conical shape (similar to the shape of a vuvuzela) wherein the shape further includes a central tubular orifice that in centered about the delivery catheter 110. In this respect, when resheathing the catheter 110, the locking tube aligns the catheter 110 to be resheathed in the protective sheath 195 in conjunction with the sheath outer component 197 that “zips” the sheath 195 up around the catheter 110 when the catheter 100 and sheath 195 enter the orifice of the sheath outer component 197 (described in more detail with respect to FIG. 3). Similarly, when unsheathing the catheter 110, the locking tube 196 maintains alignment of the catheter 110 to be unsheathed from the protective sheath 195 such that the larger end the conical locking tube 196 (e.g., the proximal end) biases the sheath 195 apart (e.g., “unzips” the protective sheath 195) to leave the catheter 110 exposed at the distal end of the locking tube 196.

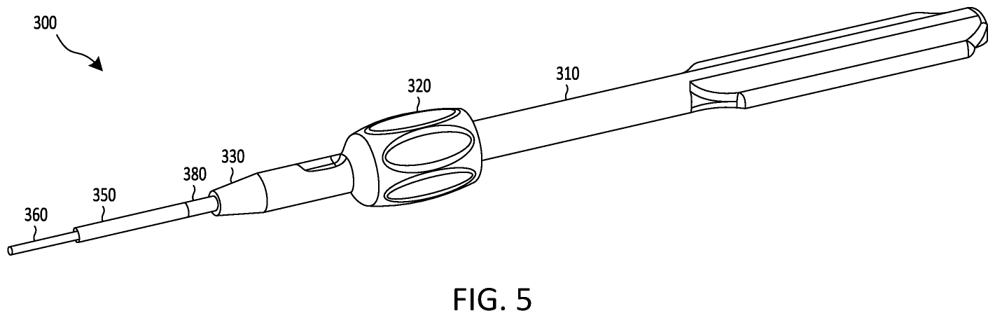

Together, the locking tube 196 and the outer component 197 facilitate the unsheathing of the catheter at a point that is separate form and outside of any actuation handle (discussed below with respect to FIG. 5) of the overall embolic device delivery system 100. Thus, the catheter 110, which is prone to damage and disfigurement if bent sharply or encounters curvilinear motion having too small of a bending radius, is prevented from encounter such a situation by the combination of the locking tube 196 and the outer component 197. This is an advantage over conventional solutions that force the catheter 110 to maneuver through a sharply angled orifice disposed in the handle.

FIG. 3 shows a more detailed diagram of a portion of an embolic device delivery system 100 showing a catheter 110 in a state of resheathing/unsheathing according to an embodiment of the subject matter disclosed herein. In FIG. 3, one can see a more detailed view of the orifice of the sheath outer component 197 as well as the sheath 195. The sheath outer component 197 comprises a cylindrical/tubular shape with a tubular cavity/orifice wherein the sheathed delivery catheter 110 (e.g., the catheter 110 zipped up inside the sheath 195) may be disposed inside the tubular cavity of the sheath outer component 197. In this configuration, the sheath outer component 197 is configured to prevent the locking tube 196 (not shown in FIG. 3) from sliding beyond a point where the reusable sheath 195 surrounds (e.g., zips up around) the catheter 110. As discussed next, there are features of the sheath outer component 197 that assist with facilitating the sheathing and unsheathing of the catheter 110.

In a first feature, the cylindrical body of the sheath outer component 197 comprises a distal end that includes a first surface 198 and a second surface 199. The surfaces are disposed at planar angle with respect to each other such that the first surface 198 is disposed in a first plane and the second surface 199 is disposed in a second plane different from the first plane. Further, the sheath outer component 197 comprises a notch 192 disposed protruding into the tubular. In an embodiment, the notch is disposed along the entire longitudinal axis of the tubular aperture. In another embodiment, the notch is only disposed at the distal end of the sheath outer component adjacent to the second surface 199. The notch 192 biases the sheath 195 into a resheathed or “zipped up” state while also preventing the locking tube 196 from locking up movement at the zipperlike resheathing of the catheter 110 (and perhaps a still attached embolic device).

As mentioned previously, the sheath 195 may be zippered and unzippered due the presence of biases crescent-shaped notches 191 that are shown in a blow-up bubble in FIG. 3. Thus, the reusable sheath 195 further comprises a tubular aperture having a longitudinal separation 193 configured to be biased in a closed position while sheathed. That is, the longitudinal separation 193 comprises a first half of a crescent-shaped notch 191a emanating from a first portion of the reusable sheath 195 and a second half of a crescent-shaped notch 191b emanating from a second portion of the reusable sheath. The first half 191a and the second half 191b are biased toward each other, and when nested, the crescent-shaped notches 191a and 191b are biased to hold the reusable sheath 195 in a closed position.

FIGS. 4(a)-(c) are diagrams of an embolic device delivery system 100 according to an embodiment of the subject matter disclosed herein. FIG. 4(a) shows an embolic device 160 coupled to a delivery catheter 110 in one embodiment. The delivery catheter 110 may include a proximal end 112 and distal end 120 along an axis (not shown) of the delivery catheter 110. When the distal end 120 of the delivery catheter 110 is inserted into an artery, the distal end 120 may be navigated through an artery to reach a desired location (e.g., a location of the aneurysm). Because the distal end 120 of the delivery catheter 110 is navigated through an artery, the embolic device 160 should be firmly coupled to the distal end 120 of the delivery catheter 110. The delivery catheter 110 may be designed as an elongated cylinder with a hollow interior tube extending from the proximal end 112 to the distal end 120. Since the delivery catheter 110 maneuvers through an artery, flexible materials may be used for the delivery catheter 110 as an elongated cylinder. In one embodiment, the flexible materials for the delivery catheter 110 may include a silicone, polyurethane (PU), polyethylene (PE), polyvinylchloride (PVC), polytetrafluoroethylene (PTFE), Polyetheretherketone (PEEK), nylon, as well as metallic catheter components, such as helical hollow stranded tubing, and laser cut flexible tubing. The flexible materials for the delivery catheter 110 may include a helical hollow strandTM and can be obtained from Fort Wayne Metals, Fort Wayne, Iowa.

The elongated cylinder of the delivery catheter 110 further includes a coupler 130 disposed along a central axis of the delivery catheter 110. The coupler 130 may have a proximal end 132 and distal end 140. In one embodiment, the proximal end 132 of the coupler 130 may be a linear member that extends through the proximal end 112 of the delivery catheter 110. The proximal end 132 may further include a mechanism for a surgeon to actuate the coupler 130 by moving the coupler 130 backward inside the delivery catheter 110, further discussed below in FIG. 5. The linear member of the coupler 130 may also form an engagement member toward the distal end 140 of the coupler 130. In one embodiment, the engagement member 140 may be formed as a small diameter loop made of a shape-memory alloy, such as Nitinol, NiTi, or nickel titanium. The shape-memory alloy possesses super elasticity and unique memory characteristics of the original shape. Thus, the shape-memory alloy may be stretched and maintained in the stretched phase; however, once the alloy is released from the stretch, the alloy will return back to the original shape. The maneuverable engagement member 140 may be further configured to be become more/less rigid and/or more/less flaccid when exposed to heat, electricity, or physical force. As discussed with respect to FIGS. 4(b) and 4(c), this allows the coupler 130 to engage, maneuver and disengage an embolic device 160 during an embolic device delivery procedure.

The embolic device 160 coupled to the delivery catheter 110 may include an embolic device 160 configured to expand once placed at the appropriate location inside the artery or near the aneurysm. In some embodiments, the embolic device 160 may be a platinum coil. The embolic device 160 may also include a proximal end 172 and a distal end 174 and a retaining mechanism 180 may be formed at the proximal end 172 of the embolic device 170 to securely couple with the delivery catheter 110. In various embodiments, the retaining mechanism 180 may be formed as a closed ring, loop, hoop, or eyelet separately formed from the embolic device 160 and affixed at the proximal end 172 of the embolic device 160. In a further embodiment, the retaining mechanism 180 may be formed integrally with the embolic device 160. With such a proximal end 172 suited to engage a coupler 130, the retaining mechanism 180 forms an aperture 190 by which the proximal end 172 of the coupler may engage and penetrate. The retaining mechanism 180 may be made of polypropylene or a platinum filament from the primary wind of the coil. During embolic device placement and delivery, the retaining mechanism 180 (and at times, the entire embolic device 160) may be disposed inside the delivery catheter 110 near the distal end 120. Thus, the diameter of the aperture 190 and the width of the embolic device 160 may be narrower than the inside diameter of the delivery catheter 110 such that the retaining mechanism 180 and embolic device 160 are held inside the distal end 120 of the delivery catheter 110 while being maneuvered through an artery.

When the delivery catheter 110 engages with the embolic device 160, the maneuverable engagement member 140 of the coupler 130 engages with the retaining mechanism 180 at the distal end 120 of the delivery catheter 110 by extending the maneuverable engagement member 140 into the aperture 190 of the retaining mechanism 180. For this configuration, the inside diameter of the aperture 190 may be slightly wider than the diameter of the maneuverable engagement member 140 such that the retaining mechanism 180 allows a small amount of movement for the maneuverable engagement member 140 to move around the inside of the aperture 190 of the retaining mechanism 180. In one embodiment, the maneuverable engagement member 140 may be extended upwardly through the aperture 190 by taking an upwardly curved shape. The maneuverable engagement member 140 may be extended downwardly or sideways instead of upwardly in response to rotation of the delivery catheter 110 due to manipulation of the delivery catheter by a surgeon such that a person having an ordinary skill in the art would change the direction of the curves accordingly. In a further embodiment, the maneuverable engagement member 140 maneuver away from the axis of the delivery catheter 110. Due to the super elasticity and shape memory characteristics of the maneuverable engagement member 140, the maneuverable engagement member 140 is capable of deforming its shape, such as from a straight configuration to an upwardly curved shape. In a further embodiment, the maneuverable engagement member 140 may be bent vertically at one portion to extend through the aperture 190 of the retaining mechanism 180.

As discussed briefly above, the delivery catheter 110 forms an upper locking window 150 on one side of the interior wall of the hollow tube near the distal end 120 of the delivery catheter 110 and a lower locking window 152 on the other side of the interior wall of the hollow tube near the distal end 120 of the delivery catheter 110. In one embodiment, the maneuverable engagement member 140 may form a U-shaped curve 154 and the downward curve 154 of the maneuverable engagement member 140 may be maintained with the locking features by the upper locking window 150 and the lower locking window 152. In this configuration, the bottom of the downward curve 154 of the maneuverable engagement member 140 may be maintained within the lower locking window 152 and the tip 200 of the maneuverable engagement member 140 may be maintained within the upper locking window 150 within the delivery catheter 110 while navigating the delivery catheter 110 into an artery. In another embodiment, the upper locking window 150 is located nearer to the distal end 120 of the delivery catheter 110 than the lower locking window 152 is to the distal end 120 of the delivery catheter 110 such that the maneuverable engagement member 140 is locked with the upper locking window 150 and the lower locking window 152 at the distal end 120 of the delivery catheter 110.

When the tip 200 of the maneuverable engagement member 140 passes through the lower locking window 152 and reaches the upper locking window 150, the maneuverable engagement member 140 further curves up such that the tip 200 of the maneuverable engagement member 140 extends through the upper locking window 150. In a further embodiment, the maneuverable engagement member 140 may bend vertically to extend through the upper locking window 150 as well. Once the maneuverable engagement member 140 is shaped in the upwardly curved position, the maneuverable engagement member 140 maintains its shape until any physical force is applied to the maneuverable engagement member 140. The upwardly curved shape of the maneuverable engagement member 140 may be formed by physically bending the maneuverable engagement member 140, such as by hand, or by maneuvering the distal end 120 of the coupler 130 to extend the maneuverable engagement member 140 through the aperture 190 such that the straight original configuration is deformed into the curved shape. In various embodiments, the upper locking window 150 and lower locking window 152 may be formed as a rectangular shape, elliptical shape, oval shape, or round shape. In a still further embodiment, the width of the locking window 150 may be slightly wider than the width of the tip 200 of the maneuverable engagement member 140. As such, the inside of the locking window 150 allows limited movement of the tip 200 to move around such that the tip 200 is secured in the locking window 150.

In addition to the locking mechanisms by the upper and lower locking windows 150, 152, a cross bar 156 extending perpendicular to the axis of the hollow tube of the delivery catheter 110 may further limit the movements of the coupler 130 both in the distal direction 140 and proximal direction 132. When the embolic device 160 is in a position coupled to the delivery catheter 110 (see FIG. 4(a)), the coupler 130 may be slid toward the distal direction. However, during the sliding, the curve of the maneuverable engagement member 140 contacts with the cross bar 156 and prevents further movement in the distal direction. Further, when the coupler 130 moves proximally, the retaining mechanism 180 and maneuverable engagement member 140 may contact the cross bar 156 such that further movement in the proximal direction 132 may be prevented.

FIG. 5 shows an actuation mechanism or handle 300 connected to a proximal end 112 of an embolic device delivery system 100 shown in FIGS. 4(a)-(c) according to an embodiment of the subject matter disclosed herein. The actuation handle 300 for maneuvering the coupler 130 and the delivery catheter 110 to release an embolic device 160 from the coupler 130 described in FIGS. 4(a)-2(c). The actuation handle 300 may be any suitable means by which a surgeon may easily maneuver the coupler 130 in the lineal direction within an artery of a patient. In one embodiment, the handle 300 is a mechanical handle 250 that can pull the coupler in the distal direction. In FIG. 5, the actuator handle 300 is shown including a distal member 310, proximal member 330, rotating barrel 320, outer shaft 350, and inner shaft 360. Those components 310, 320, 330, 350, and 360 are coupled each other. In another embodiment, an adhesive may be placed between outer shaft 350 and proximal member 330 such that the outer shaft 370 is stably fixed to the proximal member 330. The actuator handle 330 is designed for a surgeon to hold the distal member 310 in his/her hand such that the rotating barrel 320 can be held by a forefinger and thumb of the surgeon to rotate in right or left directions. In one embodiment, rotating the rotating barrel 320 in the left direction may extend the inner shaft 360 to the proximal direction and rotating the rotating barrel 320 in the right direction may shorten the inner shaft 360 in the distal direction.

Referring back to FIG. 4(a)-(c), FIG. 4(b) shows the embolic device 160 in a position to be released from the delivery catheter 110 according to an embodiment of the subject matter disclosed herein. When the delivery catheter 110 reaches the desired location (e.g. an aneurysm), the release of the maneuverable engagement member 140 may be actuated by a surgeon by pulling the linear member of the coupler. In this embodiment, the release of the maneuverable engagement member 140 occurs when the proximal end 132 of the coupler 130 is pulled toward the proximal end 112 of the delivery catheter 110. Then, the downward curve 154 of the maneuverable engagement member 140 may be pulled up from the lower locking window 152 and the tip 200 of the maneuverable engagement member 140 may be simultaneously pulled down from the locking window 150. The tip 200 of the maneuverable engagement member 140 may be further pulled down through the aperture 190 of the retaining mechanism 180 of the embolic device 160 and the downward curve 154 of the maneuverable engagement member 140 is completely taken out from the lower locking window 152. While the maneuverable engagement member 140 passes through the aperture 190, an edge 210 of the retaining mechanism 180 presses the upwardly curved or bent portion of the maneuverable engagement member 140 and a lower side of the cross bar 156 to make the curved or bent portion slightly straight such that the maneuverable engagement member 140 may be easily pulled out from the aperture 190. When the tip 200 of the maneuverable engagement member 140 passes through the lower of the cross bar 156, the cross bar 156 further pushes the upwardly curved or bent portion down, such that the tip 200 becomes straighter. This will help the maneuverable engagement member 140 to be pulled clearly inside of the delivery catheter 110 without dragging or scratching the inside wall of the catheter 110.

FIG. 4(c) shows the embolic device 160 being completely disengaged from the delivery catheter 110 in one embodiment. When the coupler 130 is pulled proximally and once the tip 200 of the maneuverable engagement member 140 is pulled out from the aperture 190 of the retaining mechanism 180, the embolic device 160 is disengaged from the distal end 120 of the delivery catheter 110. Then, the surgeon may carefully remove the entire delivery catheter 110 by pulling the delivery catheter 110 out from the artery to complete the procedure. In such a removal maneuver, the catheter may be resheathed inside the protective sheath 195 for further use when a new embolic device may be engaged. Alternatively, if the embolic device remains attached to the coupling mechanism, the entire embolic device delivery system 100 may be set back to an initial deployment state where the catheter 110 is resheathed and ready for next use.

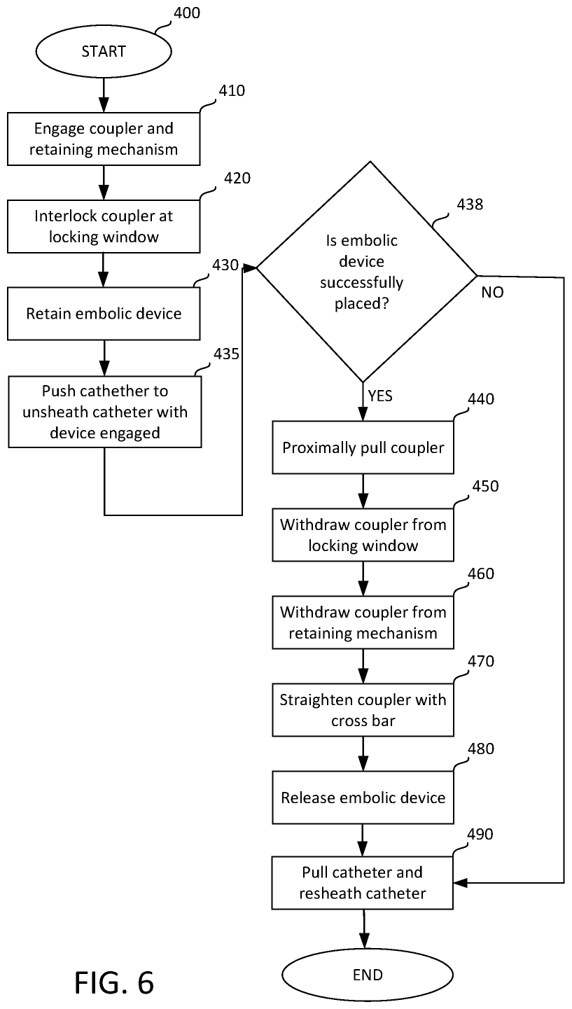

FIG. 6 is a flowchart for illustrating a method for sheathing and unsheathing an embolic device delivery system according to an embodiment of the subject matter disclosed herein. Prior to insertion into any artery, the embolic device 160 may be engaged with the delivery catheter 110 by engaging the maneuverable engagement member 140 of the coupler 130 with the aperture 190 of the retaining mechanism 180 (step 410). The maneuverable engagement member 140 of the coupler 130 forms a curved shape and further extends into the upper locking window 150 and lower locking window 152 of the delivery catheter 110, such that the coupler 130 secures the embolic device 160 with the delivery catheter 110 (step 420). As such, the embolic device is now retained by the coupling mechanism (step 430). The delivery catheter 110 is then unsheathed (step 435) and inserted into an artery and navigated to the desired location of the artery with the embolic device 160 retained by the delivery catheter 110. The unsheathing (step 435) may be accomplished by maneuvering the catheter 110 out of the reusable sheath 195 by moving the catheter 110 in a distal direction (e.g., rotating an actuating portion of the handle) while holding the outer sheath component 197 stationary.

When the delivery catheter 110 reaches the desired location, a physician may determine at step 438 whether or not the embolic device is located in a proper position for deployment. If YES, then the proximal end of the coupler 130 is pulled proximally (step 440). By pulling, the maneuverable engagement member 140 of the coupler 130 is withdrawn from the upper locking window 150 and lower locking window 152 (step 450). By further proximally pulling, the maneuverable engagement member 140 is further withdrawn from the aperture 190 of the retaining mechanism 180 (step 460). When the tip 200 of the maneuverable engagement member 140, especially the curved shape of the maneuverable engagement member 140 contacts a cross bar 156, the cross bar 156 pushes the maneuverable engagement member 140 down, such that the tip 200 is not dragged or scratched within the delivery catheter 110 (step 470). Once the tip 200 of the maneuverable engagement member 140 is completely withdrawn from the aperture 190, the embolic device 160 is released from the delivery catheter 110 and the delivery catheter is withdrawn from the artery (step 480).

If, however, at step 438, the embolic device is not properly placed according to a physician's opinion (e.g., the NO branch), the embolic device may be retrieved while still attached to the coupling mechanism at the distal end of the catheter 110 and the catheter 110 may be resheathed along with the embolic device for another use. Such a resheathing process at step 490 may be accomplished by securing the outer sheath component 197 in a fixed position while maneuvering a catheter 110 in a proximal direction. This will facilitate the nesting of the first V-shaped portion (see FIG. 3) of the reusable sheath 195 with the second V-shaped portion (also see FIG. 3) at a point where the catheter 110 proximally moves through the tubular aperture of the outer sheath component 197. The nesting is further facilitated by the locking tube 196 disposed about an unsheathed portion of the catheter 110. In this manner, the locking tube 196 is prevented from moving beyond the outer sheath component 197 by the notch 193 disposed in the tubular aperture of the outer sheath component 197. The method may then end once the catheter 110 is resheathed whether or not the embolic device is still attached.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely intended to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

What is claimed is:

1. A device for use in an embolic procedure, the device comprising:
a catheter having a coupler disposed therein and configured to engage an embolic device;
a reusable sheath configured to surround a portion of the catheter and embolic implant in a sheathed state and configured to reveal the embolic implant and the portion of the catheter in an unsheathed state;
a sheath outer component slidably coupled to the reusable sheath and configured to bias the sheath to a sheathed state surrounding the catheter; and
a locking tube slidably coupled to the catheter and configured to bias the catheter for unsheathing.

2. The device of claim 1, wherein the sheath outer component further comprises a cylindrical body having a tubular aperture suited to aid the sheath to engage the reusable sheath.

3. The device of claim 2, wherein the cylindrical body comprises a first end face and a second end face such that at least one end face includes a first surface disposed in a first plane and a second surface disposed in a second plane different from the first plane.

4. The device of claim 1, wherein the sheath outer component further comprises a cylindrical body having a tubular aperture suited to engage the catheter, the tubular aperture further comprises a notch protruding into the tubular aperture.

5. The device of claim 1, wherein the catheter comprises a locking window disposed on the catheter and configured to engage the coupler at the locking window.

6. The device of claim 5, wherein the coupler comprises an elastic coupler configured to be manipulated to engage the embolic device while also engaging the locking window.

7. The device of claim 1, wherein the reusable sheath comprises a tubular aperture having a longitudinal separation configured to be biased in a closed position while sheathed.

8. The device of claim 7, wherein the longitudinal separation comprises a first crescent-shaped notch emanating from a first portion of the reusable sheath and a second crescent-shaped notch emanating from a second portion of the reusable sheath and suited to nest with the first crescent-shaped notch, the nested v-shaped notches biased to hold the reusable sheath in a closed position when nested.

9. The device of claim 1, wherein the locking tube comprises a conical body having a tubular aperture, the tubular aperture suited to engage the catheter to the reusable introducer sheath.

10. The device of claim 1, wherein the sheath outer component is configured to facilitate resheathing of the embolic device and to prevent the locking tube from sliding beyond a point where the reusable sheath surrounds the catheter.

11. An embolic device delivery system, comprising:
an embolic device having a retainer ring;
a catheter having a coupler disposed therein and configured to engage the embolic device at the retainer ring;
a reusable sheath configured to surround the embolic device and the catheter in a sheathed state and configured to reveal the catheter in an unsheathed state;
a sheath outer component slidably coupled to the reusable sheath and configured to bias the sheath to a sheathed state surrounding the catheter; and
a locking tube slidably coupled to the catheter and configured to unsheath the catheter and to lock the reusable sheath to the embolic device and catheter.

12. The system of claim 11, wherein the sheath outer component further comprises a cylindrical body having a tubular aperture suited to engage the reusable sheath to the catheter.

13. The system of claim 12, wherein the cylindrical body comprises a first end face and a second end face such that at least one end face includes a first surface disposed in a first plane and a second surface disposed in a second plane different from the first plane.

14. The system of claim 11, wherein the sheath outer component further comprises a cylindrical body having a tubular aperture suited to engage the reusable sheath, the tubular aperture further comprises a notch protruding into the tubular aperture.

15. The system of claim 11, wherein the reusable sheath comprises a tubular aperture having a longitudinal separation configured to be biased in a closed position while sheathed.

16. The system of claim 15, wherein the longitudinal separation comprises a first crescent-shaped notch emanating from a first portion of the reusable sheath and a second crescent-shaped notch emanating from a second portion of the reusable sheath and suited to nest with the first crescent-shaped notch, the nested crescent-shaped notches biased to hold the reusable sheath in a closed position when nested.

17. The system of claim 11, wherein the sheath outer component is configured to facilitate resheathing of the reusable sheath surrounds the embolic device and the catheter.

18. A method for resheathing an embolic device delivery system, comprising:
securing an outer sheath component in a position while maneuvering a catheter in a proximal direction; and
nesting a first crescent-shaped portion of a reusable sheath a second crescent-shaped portion of the reusable sheath at a point where the catheter proximally moves through a tubular aperture of the outer sheath component, the nesting facilitated by a locking tube disposed about an unsheathed portion of the catheter;
wherein the locking tube is prevented from moving beyond the outer sheath component by a notch disposed in a tubular aperture of the outer sheath component.

19. The method of claim 18, further comprising biasing the reusable sheath in a closed position when the first crescent-shaped portion of the reusable sheath is nested with the second crescent-shaped portion of the reusable sheath.

20. The method of claim 18, further comprising maneuvering the catheter out of the reusable sheath by moving the catheter in a distal direction while holding the outer sheath component stationary.
